(19) United States
(12) Patent Application Publication
(10) Pub. No.: US 2020/0359899 A1
(43) Pub. Date: Nov. 19, 2020

(54) METHODS AND COMPUTER PRODUCT FOR IDENTIFYING TISSUE COMPOSITION USING QUANTITATIVE MAGNETIC RESONANCE IMAGING (qMRI)

(71) Applicant: Aviv MEZER, Jerusalem (IL)

(72) Inventor: Aviv MEZER, Jerusalem (IL)

(21) Appl. No.: 16/960,622

(22) PCT Filed: Jan. 8, 2019
(86) PCT No.: PCT/IL2019/050041

§ 371 Date: Jul. 8, 2020

Related U.S. Application Data
(60) Provisional application No. 62/614,563, filed on Jan. 8, 2018.

(51) Int. Cl. A61B 5/00; A61B 5/055; G01R 33/48; G01R 33/44; G01R 33/56

(52) U.S. Cl. CPC A61B 5/0042; A61B 5/055; G01R 33/5605; G01R 33/448; G01R 33/4828

(57) ABSTRACT

In accordance with some embodiments of the invention there is provided a method for quantification of molecular composition of a scanned tissue based on an MRI signal including receiving qMRI (quantitative MRI) data from a tissue scan, the qMRI data comprising at least a first and a second parameter, wherein said first parameter is a non-water fraction of the scanned tissue, calculating a dependency of the second qMRI parameter on a non-water fraction of the scanned tissue, and quantifying a molecular composition of the scanned tissue based on the calculation. In accordance with some embodiments of the invention there is provided a computer program product comprising a non-transitory computer-readable storage medium having program code embodied therewith, the program code executable by at least one hardware processor to carry out the method.

### CROSS-REFERENCE TO RELATED APPLICATIONS

This application claims the benefit of U.S. Provisional Patent Application No. 62/614,563 filed Jan. 8, 2018, entitled: DISENTANGLING THE CONTRIBUTIONS OF BRAIN TISSUE FRACTION AND COMPOSITION TO QUANTITATIVE MRI, the contents of which are incorporated herein by reference in their entirety.

### FIELD OF THE INVENTION

The present invention, in some embodiments thereof, relates to methods of tissue fractions identification and, more particularly, but not exclusively, to tissue fraction identification using quantitative MRI.

### BACKGROUND

Magnetic resonance Imaging (MRI) is a most prominently imaging technique used in diagnostic medicine and biomedical research, it also may be used to form images of non-living objects. MRI, being a non-invasive scanning technique, is commonly used to visualize internal organs in the body without exposure to ionizing radiation.

However, MRI scans are capable of producing a variety of chemical and physical data, in addition to detailed spatial images. The term quantitative MRI (qMRI) refers to the use of an MRI as a scientific tool, for example by obtaining such data.

The concept of a qMRI being a scientific measuring instrument has similarity to that of a blood pressure measurement. There are three main and important issues in the context of good and reliable clinical qMRI research: the sensitivity, uniqueness, and accuracy of the indicator used to characterize the biological tissue.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with some embodiments, a method for quantification of molecular composition of a scanned tissue based on an MRI signal including receiving qMRI (quantitative MRI) data from a tissue scan, the qMRI data including at least a first and a second parameter, wherein the first parameter is a non-water fraction of the scanned tissue, calculating a dependency of the second qMRI parameter on a non-water fraction of the scanned tissue and quantifying a molecular composition of the scanned tissue based on the calculation. In some embodiments, the tissue is brain tissue.

According to some embodiments, the method includes associating molecular composition of the scanned tissue with at least one of a specific region of the brain, a specific pathology of the brain tissue and an age of the scanned tissue. In some embodiments, eliminating the confounding effect of water on the qMRI parameters by the evaluating a dependency of the qMRI parameters on a non-water fraction of the scanned tissue. In some embodiments, the obtained qMRI parameters include at least one of T1, T2, R1, R2, MT, MTV, susceptibility and CEST. In some embodiments, the non-water fraction of the scanned tissue is at least Proton Density (PD).

According to some embodiments, quantifying the MTV to overcome the confounding effect of water content on qMRI parameters and deriving therefrom tissue-specific properties. In some embodiments, the method includes generating for at least one brain area of different brain areas a linear fit the slope of which represents MTV derivatives of R1. In some embodiments, the method includes In some embodiments, the method includes generating for at least one brain area of different brain areas a linear fit the slope of which represents MTV derivatives of MTsat. In some embodiments, the method includes associating changes in the non-water fraction with changes in a molecular composition of a tissue being scanned.

According to some embodiments, the tissue is brain tissue and the method includes associating changes in the non-water fraction with specific regions of the brain being scanned. In some embodiments, the method includes associating changes in the non-water fraction with change in age of the tissue being scanned. In some embodiments, the tissue is brain tissue and the method includes generating a plurality of a unique tissue signatures for corresponding brain regions over a period of time and measuring for each signature the dependency of qMRI parameters on MTV. In some embodiments, the tissue is brain tissue and the method includes combining local dependencies of the different qMRI parameters on MTV and generating a unique signature for at least one corresponding brain region from the different brain regions.

According to some embodiments, the method includes identifying and classifying aberrant scan signatures of specific brain regions as qMRI scan signatures corresponding to specific pathologies in the specific brain regions. In some embodiments, the method includes identifying and classifying tissue qMRI scan signatures associated with at least one qMRI parameter.

In accordance with an some embodiments of the invention there is provided a method for predicting the molecular composition of the human brain, the method including estimating a matrix Mpure for a selected region of brain tissue, measuring MDM measurement of lipid mixture in the region and deriving Mmix where Mmix is a vector of the MDM measurements, such that:

[Mₘᵢₓ]=[Mₚᵤᵣₑ]*[F]

wherein, in some embodiments, F is a vector of the lipid fractions of the mixture, and Mpure is a matrix of the MDM measurements of the pure lipids, and estimating the lipid composition of the brain tissue in the brain segment by calculating:

[F]=[Mpure]−1*[Mmix]

wherein the calculation includes at least one of human brain molecular features: % PE, % PS, % PtdCho % PI, % Spg, phospholipids/proteins and phospholipids/cholesterol. In some embodiments, the calculation includes at least one of MDM measurements: dR1/dMTV, dMTsat/dMTV, dR2dMTV, dMD/dMTV.

In accordance with some embodiments of the invention the method includes identifying a plurality of molecular features with the largest loadings on the first PC of molecular variability.

In accordance with an some embodiments of the invention there is provided a computer program product including a non-transitory computer-readable storage medium having program code embodied therewith, the program code executable by at least one hardware processor to receive qMRI (quantitative MRI) data from a tissue scan, the qMRI data including at least a first and a second parameter, wherein the first parameter is a non-water fraction of the scanned tissue, calculate a dependency of the second qMRI parameter on a non-water fraction of the scanned tissue, and quantify a molecular composition of the scanned tissue based on the calculation. In accordance with some embodiments the tissue is brain tissue. In accordance with some embodiments the second qMRI parameter includes at least one of T1, T2, R1, R2, MT, MTV, susceptibility and CEST.

In accordance with some embodiments the computer program product generates a plurality of a unique tissue signatures for corresponding brain regions over a period of time and measures for each signature the dependency of qMRI parameters on MTV. In accordance with some embodiments the tissue is brain tissue and the computer program product combines local dependencies of the different qMRI parameters on MTV and generates a unique signature for at least one corresponding brain region from the different brain regions. In accordance with some embodiments the computer program product analyzes the data and identifies and classifies aberrant scan signatures of specific brain regions as qMRI scan signatures corresponding to specific pathologies in the specific brain regions.

In accordance with some embodiments of the invention, the computer program product analyzes the data and identifies and classifies tissue qMRI scan signatures associated with at least one qMRI parameter. In accordance with some embodiments the non-water fraction of the scanned tissue is at least Proton Density (PD). In accordance with some embodiments, the computer program product quantifies the MTV to overcome the confounding effect of water content on qMRI parameters and derives therefrom tissue-specific properties. In accordance with some embodiments the computer program product generates for at least one brain area of different brain areas a linear fit the slope of which represents MTV derivatives of R1

In accordance with some embodiments of the invention, the computer program product generates for at least one brain area of different brain areas a linear fit the slope of which represents MTV derivatives of MTsat. In some embodiments, the computer program product associates changes in the non-water fraction with changes in a molecular composition of a tissue being scanned. In some embodiments, the tissue is brain tissue and the computer program product associates changes in the non-water fraction with specific regions of the brain being scanned.

In accordance with some embodiments, the computer program product associates changes in the non-water fraction with change in age of the tissue being scanned. In some embodiments, the computer program product identifies types of lipids using for MT the mathematical expression:

$${MTnorm}_{mixture} = {\sum }_{i = 1}^{l} ( {slopemt}_{lipi} ) \cdot ( WFi ) + {intercept}_{lipi} \cdot ( WFi )$$

In accordance with some embodiments of the invention there is provided a method for quantification of molecular composition of a scanned tissue including receiving qMRI (quantitative MRI) data from a tissue scan, the qMRI data including at least a first and a second parameter, wherein the first parameter is proton density (PD) of the scanned tissue, generating multi-parametric mapping of the second parameter based on a local linear dependency of the second qMRI parameter on PD, and generating a unique signature corresponding to the tissue region. In some embodiments, the second qMRI parameter include at least one of T1, T2, R1, R2, MT, MTV, susceptibility and CEST.

In addition to the exemplary aspects and embodiments described above, further aspects and embodiments will become apparent by reference to the figures and by study of the following detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a top view simplified illustration of a T1 map (msec) of a selected qMRI slice of sample phantoms in accordance with some embodiments of the invention;

FIGS. 2A and 2B are graph representations of correspondence of PD with an estimation of WF (FIG. 2A) and correspondence of phosphatidylserine (PS) liposome with an estimation of WF (FIG. 2B) in accordance with some embodiments of the invention;

FIGS. 3A and 3B are graph representations of R1 (1/T1) (FIG. 3A) and MTnorm (FIG. 3B) linear dependency on WF and comparison of slopes of the dependencies to separate between PS and PC in accordance with some embodiments of the invention;

FIGS. 4A-4C are graph representations of scanning results of phantoms with varying water concentration and different lipid content in accordance with some embodiments of the invention;

FIGS. 5A-5B are graph representations of correlation of the molecular variability with standard qMRI parameters and MTV in accordance with some embodiments of the invention;

FIGS. 6A and 6B are graph representations of linearity of the dependency of R1 (FIG. 6A) and MTsat (FIG. 6B) on MTV in the human brain, the linear relationship between MTsat to MTV in different brain regions of a single subject in accordance with some embodiments of the invention;

FIGS. 7A-7C are Mill image scans derived representative R1 (upper map) and MTV (lower map) maps (FIG. 7A) and graphs (FIGS. 7B and 7C) demonstrating R1 dependency on MTV for different brain regions in the left hemisphere of a human brain in accordance with some embodiments of the invention;

FIGS. 8A-8C, are MRI image scans derived representative MTsat and MTV maps (FIG. 8A) and graphs (FIGS. 8B and 8C) demonstrating MTsat dependency on MTV for different brain regions in the left hemisphere of human brain in accordance with some embodiments of the invention;

FIG. 9 is a graph representing quantification of unique signatures of different brain regions in accordance with some embodiments of the invention;

FIGS. 10A-10C are graph representations of changes with age in the unique brain region signatures in accordance with some embodiments of the invention;

FIGS. 11A-11C are signature graphs representations of a comparison of MTsat to MTV slope (FIG. 11A), MTV (FIG. 11B) and MTsat (FIG. 11C) in the Thalamus; and

FIGS. 12A-12C are signature graphs representations of a comparison MTsat to MTV slope (FIG. 12A), MTV (FIG. 12B) and MTsat (FIG. 12C) in the Cortex.

### DETAILED DESCRIPTION

### Glossary

MDM Multidimensional dependency on MTV

MTV Lipid and macromolecular tissue volume

PC Principal component

PS Phosphatidylserine

PtdCho Phosphatidylcholine

PtdCho-Chol Phosphatidylcholine-cholesterol

PI-PtdCho Phosphatidylinositol-phosphatidylcholine

Spg Sphingomyelin

Spoiled5GE Spoiled Gradient Echo

SEIR Spin Echo Inversion Recovery

PD Proton Density

R1 =1/T1 the longitudinal relaxation rate in units of sec-1

MTV Macromolecular Tissue Volume

NMR Nuclear Magnetic Resonance (Imaging)

MT Magnetic Transfer

qMT quantitative magnetic transfer

CEST Chemical exchange saturation transfer

According to an aspect of some embodiments of the present invention there is provided a method for quantification of molecular composition of a scanned tissue based on an MRI signal. In some embodiments, molecular composition of a scanned tissue is obtained by evaluating a dependency of various qMRI parameters on a non-water fraction of tissue. In some embodiments, the qMRI parameters include one or more of T1, T2, R1, R2, MT, qMT, susceptibility, CEST and PD.

According to an aspect of some embodiments of the present invention there is provided a method for localizing regions of the nervous system by identifying a dependency of one or more qMRI parameters on a non-water fraction specific to the region of the nervous system being evaluated. In some embodiments the evaluated nervous system tissue is brain tissue.

According to an aspect of some embodiments of the present invention there is provided a method for identifying and quantifying unique signatures of different regions of the brain using qMRI-generated parameters. In some embodiments, the parameters are based on the molecular (e.g., macromolecular) non-water fraction of the tissue.

According to an aspect of some embodiments of the present invention there is provided a computer program product comprising a non-transitory computer-readable storage medium having program code embodied therewith, the program code executable by at least one hardware processor to execute at least one of the methods described herein.

Conventional MRI is based on the acquisition of contrast images. These images are affected by many different contrast mechanisms, such as the MR pulse sequence, the MR scanner settings, B0- and B1-field inhomogeneities, as well as the different tissue properties. In conventional MRI the MR scanner settings are chosen to highlight or saturate tissue properties, resulting in e.g. T1-weighted or T2-weighted images. Conversely, quantitative MRI (qMRI) is aimed at the direct measurement of physical tissue properties, such as the relaxation times, T1 and T2, as well as the proton density (PD). These properties are, in theory, independent of acquisition method and system imperfections.

In-vivo quantitative MRI (qMRI) aims at characterizing the biological properties of a tissue e.g., brain tissue, by direct measurement of the physical tissue properties. However, qMRI parameters are sensitive both to the molecular tissue properties as well as to the water content within each voxel.

As will be described in greater detail elsewhere herein, there is provided a method that approaches MRI tissue evaluation from a different angle which comprises evaluating a dependency of qMRI parameters e.g., PD or PD-derived MTV, on the non-water fraction thereby eliminating the confounding effect of water on the qMRI parameters and providing tissue-specific measurements.

As demonstrated in greater detail elsewhere herein, dependency of qMRI parameters on the non-water fraction of the tissue being scanned changes as a reflection of the associated molecular composition of the tissue being scanned.

In some embodiments, when accounting for WF, different contributions of each lipid for each qMRI parameter can be isolated and identified thereby characterizing a lipid's qMRI signature. In the human brain, for example, the method generates and identifies unique tissue qMRI scan signatures associated with different brain regions as well as region-specific age-related changes. In some embodiments, the unique tissue qMRI scan signatures differ in one or more of the qMRI parameters including one or more of T1, T2, R1, R2, MT susceptibility, CEST, and PD.

The human brain is comprised mainly of 70-80% water, 10-11% proteins and 5-15% lipids; and distribution of these and other molecules varies between different brain regions, across age groups, as well as across various pathological states. In some embodiments, a variation from a typical unique tissue qMRI scan signature of a specific brain region can indicate pathology in that specific brain region. In some embodiments, such aberrant tissue qMRI scan signatures of a specific brain region are identified and classified as qMRI scan signatures corresponding to specific pathologies and/or a pathologies specific to corresponding brain region.

Currently, localizing and quantifying these molecules in vivo, using noninvasive methods is quite challenging. Though the methods as disclosed herein focus on characterizing the structural and biological properties of brain tissue, in some embodiments, in-vivo quantitative MRI (qMRI) can be used for characterizing the structural and biological properties of other various body tissues.

Quantitative MRI (qMRI) relaxation parameters are sensitive to specific brain tissue such as myelin. Additionally, NMR studies indicate that relaxation constants are influenced by the molecular environment and can reflect lipid content. However, as water content governs MR signal intensity, qMRI relaxation parameters are influenced by both the underlying variability in water content of the tissue and the specific tissue composition. Water content can be estimated using qMRI and this measurement, along with its complementary lipid and macromolecular tissue volume (MTV), are independent of tissue composition. Nevertheless, most current studies of water relaxation time in the human brain neglect the quantification of molecular composition contributions to the MM signal.

There is therefore provided a method that exploits the MTV quantification to overcome the confounding effect of water content on qMRI parameters and reveal tissue-specific properties. To this end, the method comprises estimating a local linear dependency of qMRI parameters on MTV. In some embodiments, this estimation provides a new transformation of qMRI measurements that enhances their sensitivity to the lipid and macromolecular content as explained in greater detail elsewhere herein.

In some embodiments, the method comprises testing an expression of underlying molecular composition by parameters generated from a qMRI tissue scan. In some embodiments, the method comprises assessing the relationship between the qMRI parameters and the water fraction (WF). As disclosed elsewhere herein, the parameters comprise one or more of T1, T2, R1, R2, MT susceptibility, CEST and PD.

### A Method for Quantification of Molecular Composition of a Scanned Tissue Based on an MRI Signal.

### The Setup and Method in a Control Example (e.g., Phantoms)

Reference is now made to FIG. 1, which is a top view simplified illustration of a T1 map (msec) of a selected qMRI slice of sample phantoms. As shown in FIG. 1, the method comprises setting up an array of phantoms 102 to be scanned by an MRI.

In some embodiments, the method comprises preparing liposomes that model cellular lipid membranes. In some embodiments, the preparing of the liposomes is carried out using a thin layer evaporation-hydration technique. In some embodiments, the method further comprises preparing the phantom array 102 by placing cuvettes 104 with liposomes samples in a polystyrene container. In some embodiments, the phantoms comprise the most abundant lipids in the human brain, e.g., Phosphatidylcholine (PC), Phosphatidylethanolamine (PE), Phosphatidylserine (PS), Phosphatidylinositol (PI), Sphingomyelin (Spg) and Cholesterol.

For the purpose of simplicity, the following relationship is defined:

MTnorm=MTon/MToff

Wherein: offset=700 Hz; flip-angle=2200, where the direct effect is minimal. Both MT and qMT approaches provide similar result.

### The Method in Human Subjects:

In some embodiments, the method comprises scanning a plurality of healthy volunteers of varying ages on a 3T Mill scanner and generating multi-parametric mapping (e.g., MTV and R1, MT saturation (MTsat), R2*, R2 and diffusion imaging).

In some embodiments, the method comprises segmenting different brain regions using a brain imaging computer program and excluding voxels at the border of each region of interest (ROI) to reduce partial volume effects.

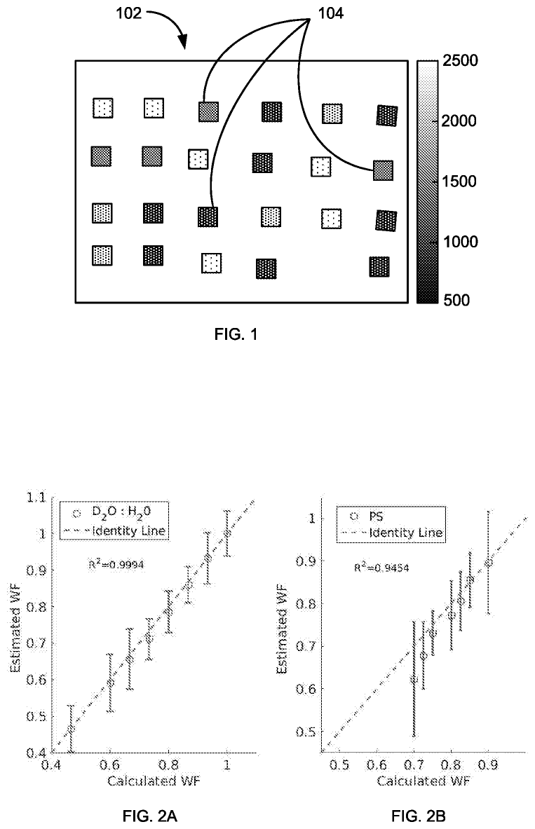

Turning now to FIGS. 2A and 2B, which are graph representations of correspondence of PD with an estimation of WF (FIG. 2A) and correspondence of phosphatidylserine (PS) liposome with an estimation of WF (FIG. 2B). The graph depicted in FIG. 2A shows an agreement of the proton density (PD)-based Mill estimation of water fraction (WF, y-axis) with the true water fraction (Calculated WF, x-axis). In the graph, each point represents the mean and standard deviation (STD) of a different sample. Each sample comprises of H2O (H-MRI visible) and D2O (H-MRI invisible) in varying volume ratios.

The graph shown in FIG. 2B demonstrates WF estimations of a phosphatidylserine (PS) liposome in different volume fractions. In this example, the calculated WF (x-axis) is shown to be in agreement with the MR-estimated WF (y-axis). In this example, the calculated WF (x-axis) was determined using the theoretical volume of a PS molecule taken from literature. The theoretical estimation does not account for water loss in the manufacturing process.

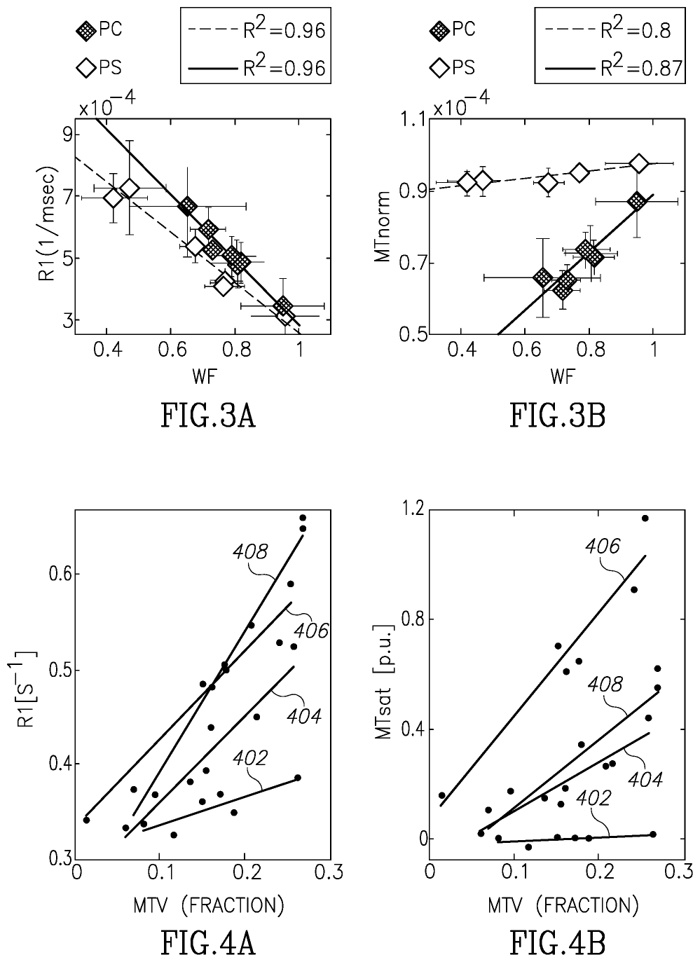

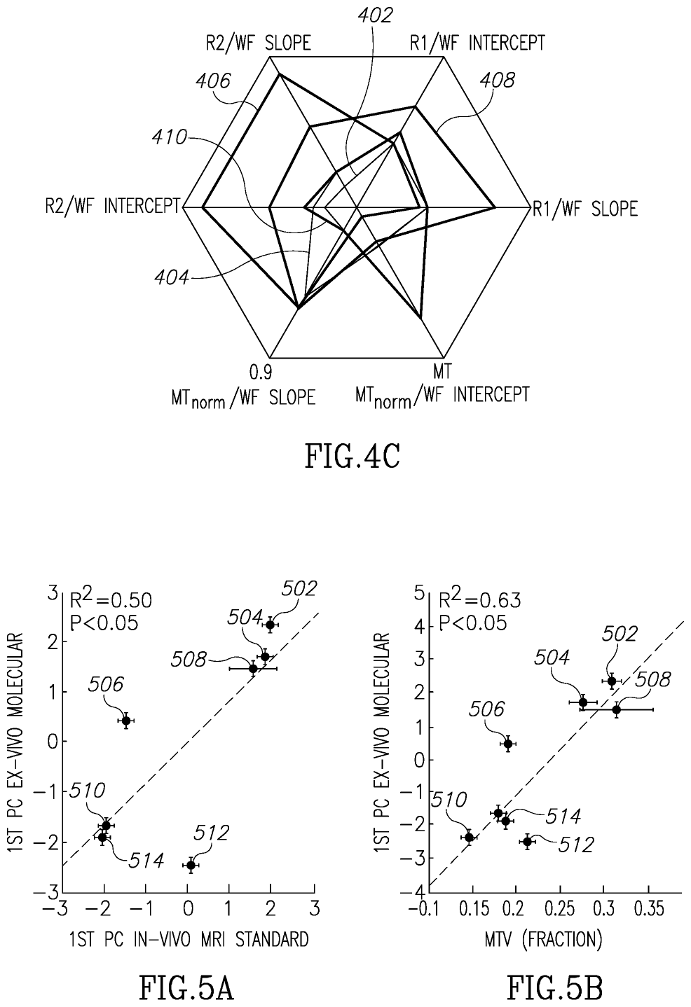

In some embodiments, and as shown in FIGS. 3A and 3B, the method comprises using R1 (1/T1) (FIG. 3A) and MTnorm (FIG. 3B) linear dependency on WF and comparing slopes of the dependencies to separate between PS and PC. As shown in FIGS. 3A and 3B, slopes of the graphs between PS and PC linear dependency on WF in both examples vary and therefore PS can be differentiated from PC. Similarly, FIGS. 4A and 4B show the linear dependency of two qMRI parameters on MTV in phantoms similar to phantoms 102 containing different phospholipids: PC 502; PC-Cholesterol 504; PS 506 and Sphingomyelin 508.

Graphs illustrated in FIGS. 4A and 4B demonstrate scanning results of phantoms 102 with varying water concentration and different lipid content (phosphatidylserine (PS 406), phosphatidylcholine (PC 402), PC-cholesterol 404 and sphingomyelin 408). The graphs show R1 (a) and MTsat (b) plotted against MTV for each lipid phantom (dots). The linear dependencies between R1 and MTsat to MTV are marked by lines. A difference between the linear dependencies is shown across phospholipids and qMRI parameters. For example, PC-cholesterol 404 and sphingomyelin 408 are to be nearly indistinguishable in terms of their MTsat slope (FIG. 4B) but appear to differ greatly in terms of their R1 slope (FIG. 4A).

In some embodiments, the method comprises fitting a representative linear slope and intercepting between the WF and the qMRI parameters across multiple scan replications. A summary of the comparison between lipids and across qMRI parameters is demonstrated graphically in FIG. 4C, which is a spiderweb plot demonstrating slopes and Intercepts representing different linear model coefficients of different phospholipids. The spiderweb plot shows the WF dependency of three qMRI parameters (R1, R2 and MTnorm) for five different lipids: PC 402; PC-Cholesterol 404; PS 406, Sphingomyelin 408 and PI-PtdCho 410. Values have been normalized such that their Euclidean norm will equal 1.

The applicants have found that MT relaxation dependency of pure PC on WF is different than those of pure PS and pure Spg. 2:1 mixtures of PC with either Spg or PS yield different results. In some embodiments, the effect of lipid mixtures is the sum of its components. Therefore, in some embodiments, the method comprises using a simple linear-weighted model and predict the result of the mixtures for the pure lipid experiments. For example, in the case of MT:

$${MTnorm}_{mixture} = {\sum }_{i = 1}^{l} ( {slopemt}_{lipi} ) \cdot ( WFi ) + {intercept}_{lipi} \cdot ( WFi )$$

Correlation of the Molecular Variability with Standard qMRI Parameters and MTV.

Reference is now made to FIGS. 5A and 5B, which are graphs that represent correlation of the molecular variability with standard qMRI parameters and MTV in accordance with some embodiments of the invention. As shown in FIG. 5A, which depicts the projection of different brain regions (WM-Frontal 502; Pons 504; Cerebellum 506; Medulla 508; Ctx-Frontal 510; Caudate Nucleus 512 and Hippocampus 514) on the 1st principal component (PC) of molecular variability (y-axis, derived from the literature) vs. their projection on the 1st principal component (PC) of standard qMRI parameters (x-axis, averaged over the young subjects). PCs were computed across seven brain regions. The correlation between the two principal components is lower than the correlation of the molecular variability with the PC of MDM.

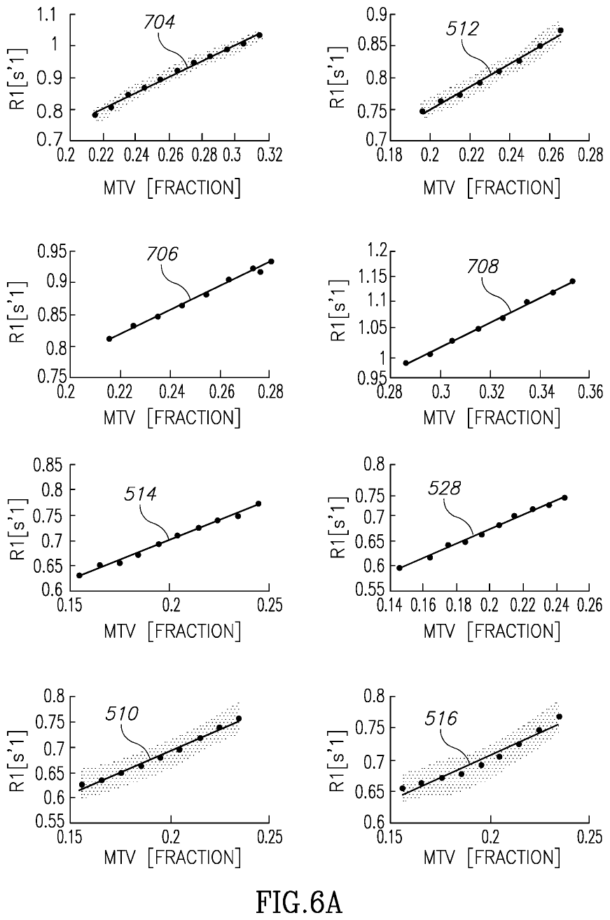

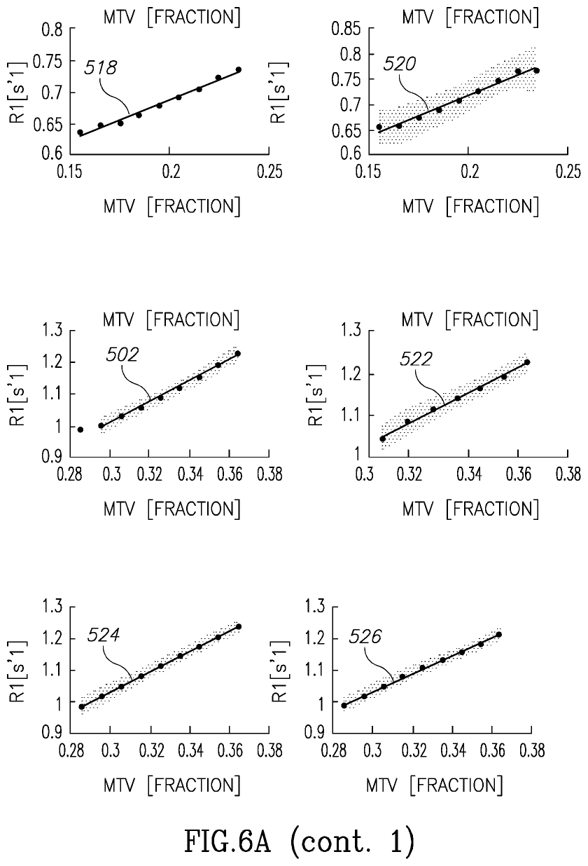

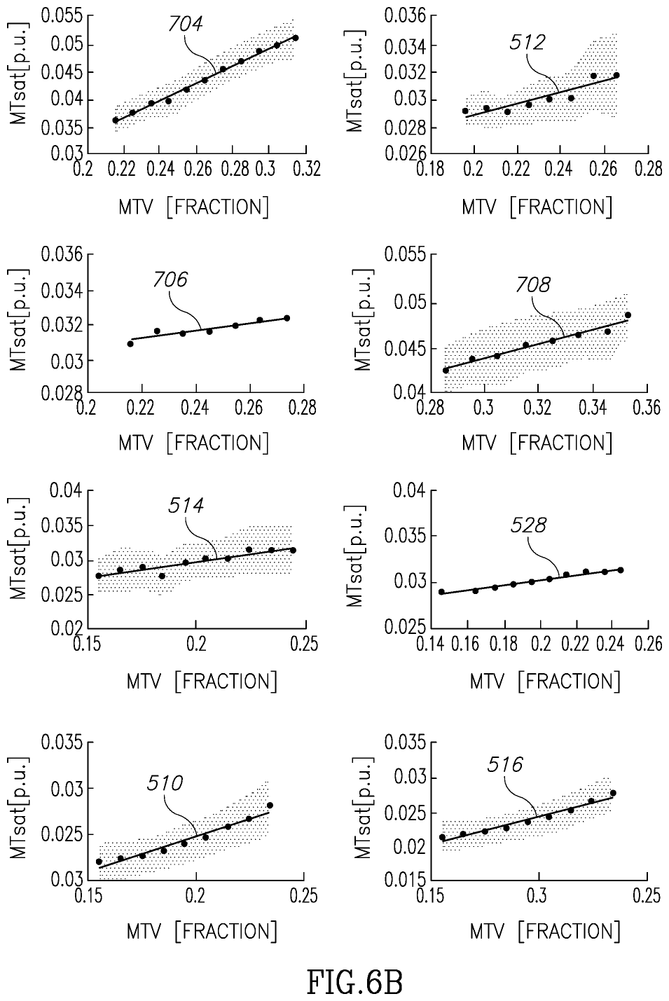

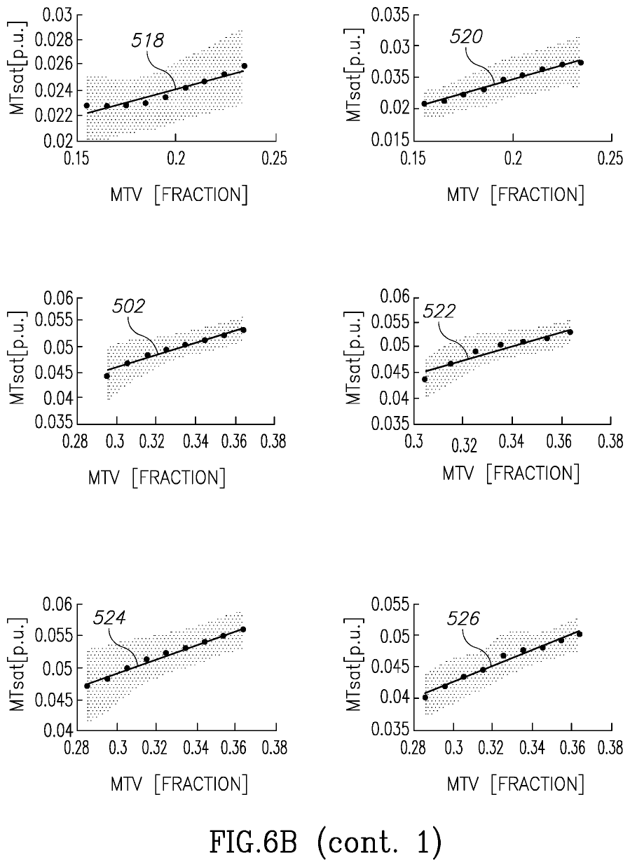

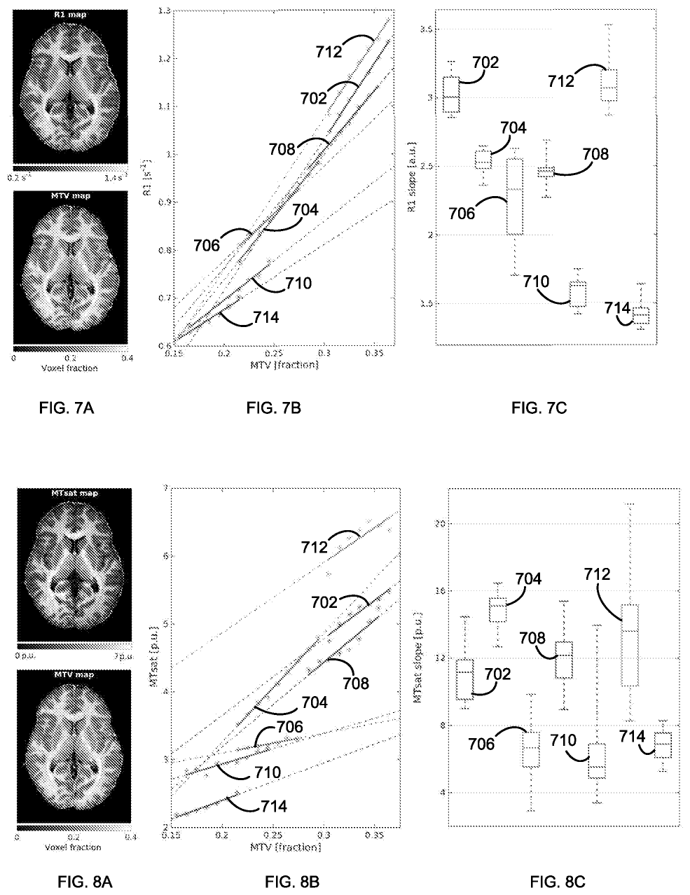

As shown in FIG. 5A, which illustrates the projection of different brain regions (WM-Frontal 502; Pons 504; Cerebellum 506; Medulla 508; Ctx-Frontal 510; Caudate Nucleus 512 and Hippocampus 514) on the 1st principal component (PC) of molecular variability (y-axis, derived from the literature) vs. their MTV values (x-axis, averaged over the young subjects). The molecular PC was computed across seven brain regions, and its correlation with MTV is lower than the correlation with the PC of multidimensional dependency on MTV (MDM) (FIG. 6B) as explained in detail elsewhere herein.

The cerebellum and the caudate nucleus have very different molecular compositions, as their projections on PC1Molecular-ex-vivo are far apart. This tissue property was not detected by conventional in-vivo qMRI methods. The two brain regions have very similar MTV (FIG. 5B), and their projections on PC1qMRI-in-vivo do not capture their molecular variability (FIG. 5A). Nonetheless, PC1MDM-in-vivo reflect the molecular variability of these two brain regions (FIG. 6B).

As shown in FIGS. 6A and 6B, which depict linearity of the dependency of R1 (FIG. 6A) and MTsat (FIG. 6B) on MTV in the human brain, the linear relationship between MTsat to MTV in different brain regions of a single subject. The graphs represent 14 brain regions: Ctx-Parietal (516); Ctx-Temporal (518); Ctx-Occipital (520); WM-Frontal (502); Wm-Parietal (522); Wm-Temporal (524); Wm-Occipital (526); Tahlamus (704); Caudate (512); Putamen (706); pallidum (708); Hippocampus (514); Amygdala (528) and Ctx-Frontal (510). The slopes of the linear fit represent the MTV derivatives of R1 (A) and MTsat (B) and are different for different brain areas.

Reference is now made to FIGS. 7A-7C, which are Mill image scans derived representative R1 (upper map) and MTV (lower map) maps (FIG. 7A) and graphs (FIGS. 7B and 7C) that demonstrate R1 dependency on MTV for different brain regions in the left hemisphere of a human brain, and FIGS. 8A-8C, which are Mill image scans derived representative MTsat (upper map) and MTV (lower map) maps (FIG. 8A) and graphs (FIGS. 8B and 8C) that demonstrate MTsat dependency on MTV for different brain regions in the left hemisphere of human brain.

The graphs depicted in FIG. 7B represent MTV values that were pooled into bins (asterisks) in different brain regions of a single subject. For each region, the linear fit between R1 and MTV was calculated and the slope was extracted.

The boxplot depicted in FIG. 7C represents slopes of different brain regions across young subjects (n=10). In each box the ticks represent the 25th, 50th and 75th percentiles; whiskers are extreme data points. In the example depicted in FIG. 7B, different brain regions show different dependencies between R1 and MTV.

The graphs depicted in FIG. 8B represent MTV values, pooled into bins (asterisks) in different brain regions of a brain of a single subject. For each region, the linear fit between MTsat and MTV was calculated, and the corresponding slope was extracted. The boxplot depicted in FIG. 8C represents slopes of the different brain regions across young subjects (n=10). In each box the ticks represent the 25th, 50th and 75th percentiles; whiskers are most extreme data points. Different brain regions show different dependencies between MTsat and MTV.

As shown in FIGS. 7A-7C and 8A-8C, in some embodiments, a linear relationship between qMRI parameters and MTV in the human brain. Different brain regions exhibit distinct dependencies on MTV, that can be quantified by the slope of the linear fit (FIGS. 7B and 8B). This slope is conserved across subjects (FIGS. 7C and 8C). Moreover, each qMRI parameter presents a different slope compared to MTV. For example, in the Putamen 706 the R1 slope is similar to the Thalamus 704 and Pallidum 708. However, their MTsat slopes are different. Other regions represented in FIGS. 7A-7C and 8A-8C include but are not limited to white matter 702, Hippocampus 514, Corpus Callosum 712 and the Cortex 714.

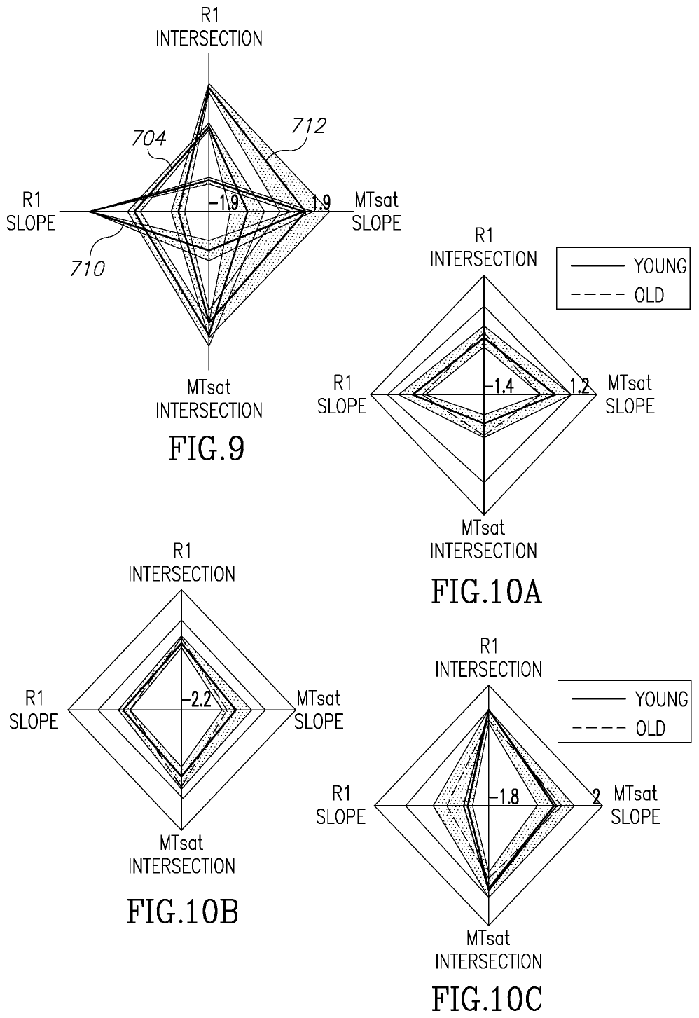

Thus, the method comprises combining local dependencies of different qMRI parameters on MTV and generating a unique signature for corresponding different brain regions. This is demonstrated in the example shown in FIG. 9, which is a graph representing quantification of unique signatures of different brain regions.

In some embodiments, the method includes for each brain region, calculating the median slope and intersection over subjects from the fit between R1 and MTsat to MTV (n=10). The spider plot shown in FIG. 9 exhibits these values (z-scored) for three example brain regions (corpus-callosum 712, thalamus 704 and hippocampus 514). Each axis generates a different separation between the regions. Together they represent a unique tissue signature for corresponding brain regions. This signature captures the contribution of the underlying tissue to the qMRI signal after accounting for the water content contribution. In some embodiments, multiple dimensions of this signature can be found with additional qMRI parameters (R2, R2* and diffusivity).

In some embodiments, the method comprises monitoring and interpretation of age-related changes in a human brain over a period of time. In some embodiments, the method comprises generating a plurality of a unique tissue signatures for corresponding brain regions over a period of time and measuring for each signature the dependency of qMRI parameters on MTV. As shown in FIGS. 10A, 10B and 10C, collectively referred to as FIG. 10, changes with age in the unique brain region signatures is demonstrated. As shown in FIG. 10, younger (under 30) brain signatures and older (over 55) brain signatures show different dependencies of qMRI parameters on MTV, and more specifically in the Putamen 706 (FIG. 10A), the Pallidum 708 (FIG. 10B) and the Corpus Callosum 712 (FIG. 10C).

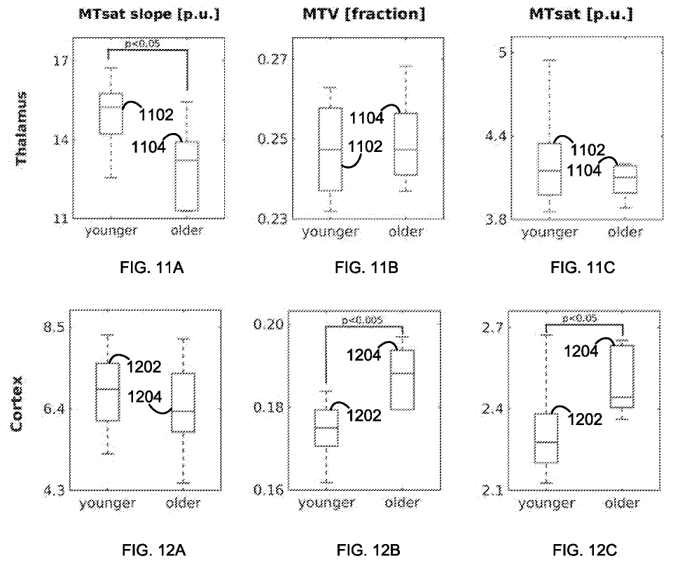

Reference is now made to FIGS. 11A, 11B and 11C, collectively referred to as FIG. 11, which are signature graphs of a comparison of MTsat to MTV slope (FIG. 11A), MTV (FIG. 11B) and MTsat (FIG. 11C) in the Thalamus between younger 1102 (under 30) to older 1104 (over 55) subjects, and to FIGS. 12A, 12B and 12C, collectively referred to as FIG. 12, which are signature graph of a comparison of MTsat to MTV slope (FIG. 12A), MTV (FIG. 12B) and MTsat (FIG. 12C) in the Cortex between younger 1202 (under 30) to older 1204 (over 55) subjects. In some embodiments, in the Thalamus, age dependent contrast is shown by the measurement of the MTsat slope. In the cortex, in some embodiments, the MTsat slope is similar between the age groups.

The cortex MTsat variation with age are explained by the MTV variation and not by tissue composition. Thus, the slope analysis allowed us to separate the contributions of the water content and the underlying tissue composition to qMRI changes measured as function of age.

### Modeling Lipid Mixtures Through Multidimensional Dependency on MTV (MDM)

In accordance with some embodiments of the invention there is hereby provided a method for modeling lipid mixtures through multidimensional dependency on MTV (MDM). In some embodiments, the method comprises predicting qMRI parameters of a lipid mixture from MDM measurements. In some embodiments, the method comprises measuring dependency of R2 on MTV for a plurality of lipids (e.g., phosphatidylcholine (PtdCho) and Phosphatidylinositol-phosphatidylcholine (PI-PtdCho). The linear relationships between R2 and MTV are fitted according to obtained data points and are marked by lines. Slopes of the marked lines represent the MTV derivatives of R2. In some embodiments, prediction is evaluated from a linear sum of the MTV dependencies of the pure lipids.

In some embodiments, the method includes obtaining MDM measurements as described elsewhere herein and predicting the qMRI parameters of a lipid mixture from the obtained MDM measurements using the following model (shown here for MTsat though predictions for R1 and R2 are done in a similar fashion):

MTsat=Σ^{L}_{i=1fi}(MTsat′ᵢ*MTV+bᵢ) 1.

Where L is the number of lipids in the mixture and fi is the fraction of the i'th lipid from the total lipid volume. MTsat′i and bi are the MDM measurements of the pure lipids. These measures were estimated from the samples prepared exclusively with each i'th lipid and were extracted from the linear fit of these samples:

MTsatᵢ=MTsat′ᵢ*MTV+bᵢ2.

Importantly, this model implies that qMRI parameters of a lipid mixture can be computed as a linear sum of qMRI parameters of the pure lipids.

Eq. 2 holds also for the qMRI parameters of lipid mixtures. Substituting this in Equation 1:

$$\begin{matrix}{MTsat}_{mix}^{′} * MTV + {B}_{mix} = {\sum }_{i = 1}^{L} {f}_{i} ( {MTsat}_{i}^{′} * MTV + {b}_{i} ) \\ = ( {\sum }_{i = 1}^{L} {f}_{i} * {MTsat}_{i}^{′} ) * MTV + {\sum }_{i = 1}^{L} {f}_{i} * {b}_{i}\end{matrix}$$

where
MTsat′mix and Bmix were extracted from the linear fit of the mixture. As this linear equation holds for large range of MTV values and due to the uniqueness of the interpolating polynomial:

MTsat′ₘᵢₓ=Σ_{i=1fi}^{L}*MTsat′ᵢ 3.

therefore, MDM measurements of a lipid mixture can be computed as the weighted sum of the MDM measurements of the pure lipids.

Note that since eq. 3 holds for the MTV derivative of several qMRI parameters, we can rewrite it in a matrix form:

[Mₘᵢₓ]=[Mₚᵤᵣₑ]*[F]4.

where Mmix is a vector of MDM measurements of a lipid mixture. F is a vector of the lipid fractions of the mixture, and Mpure is a matrix of the MDM measurements of the pure lipids. For example, a mixture of PS and Spg with ratios of 2:1 respectively can be represented by the following equation:

$$[ \begin{matrix}R {1}_{mix}^{′} \\ {MTsat}_{mix}^{′} \\ R {2}_{mix}^{′}\end{matrix} ] = [ \begin{matrix}R {1}_{PS}^{′} & R {1}_{Spg}^{′} & R {1}_{PtdChol}^{′} \\ {MTsat}_{PS}^{′} & {MTsat}_{Spg}^{′} & {MTsat}_{PtdChol}^{′} \\ R {2}_{PS}^{′} & R {2}_{Spg}^{′} & R {2}_{PtdChol}^{′}\end{matrix} ] * [ \begin{matrix}2 \\ 1 \\ 0\end{matrix} ]$$

where R1′, MTsat′, and R2′ are the derivatives of these qMRI parameters with respect to MTV (MDM).

In some embodiments, this system is extended to represent several mixtures simultaneously by adding columns describing these mixtures to [Mmix] and [F]. In this case, F is a 3×12 matrix of the lipid composition of each mixture (9 two-lipids samples, and 3 single-lipid samples). The columns of F are different mixtures, and the rows are the volume-based fractions of different lipids. Mmix is a 3×12 matrix of MDM measurements. The rows of Mmix are the MTV derivatives of R1, R2 and MTsat, and the columns are different mixtures. Mpure is a 3×3 matrix with the MDM measurements of pure lipids. The rows of Mpure are different MDM measurements, and the columns are different lipids.

MDM-based Prediction for the lipid composition of a mixture Eq. 4 can be transformed to allow prediction of the lipid fractions of a mixture from MDM measurements:

[F]=[Mpure]−1*[Mmix] 5.

A potential advantage of this model is in that this model provides a good approximation for the lipid composition of a mixture. Additionally, this model does not require prior knowledge about the specific lipid constituents.

### MDM-Based Prediction for the Molecular Composition of the Human Brain

The human brain is far more complex than a lipid mixture. As it is more difficult to a priori estimate a matrix Mpure for brain tissue, however, in some embodiments, it is possible to evaluate Mpure for brain tissue through cross-validation.

In some embodiments, the method comprises validating the approach on lipids samples and fitting Mpure in each iteration using [F] and [Mmix] of a plurality of mixtures. In some embodiments, the method comprises using the Mpure estimate to predict the fractions of the left-out (other than Mpure) mixture components. Using this process, good estimations are obtained for the composition of the lipid mixtures. Moreover, fitted elements of the Mpure matrix are similar to MDM measurements of pure lipids.

In another embodiments, the method comprises using the MDM approach to predict the molecular composition of the human brain. For this, the method comprises using equation No. 5 and the same cross-validation process; prediction for each brain region is computed by removing the selected brain region from the system and solving for the other brain regions.

In some embodiments, the calculation involves one or more human brain molecular features (% PE, % PS, % PtdCho % PI, % Spg, phospholipids/proteins, phospholipids/cholesterol), and one or more MDM measurements (dR1/dMTV, dMTsat/dMTV, dR2dMTV, dMD/dMTV). In some embodiments, the method comprises using PCA to reduce the dimensionality of the system and avoid over-fitting.

In some embodiments, the method is used for identifying a plurality (e.g., three) molecular features with the largest loadings on the first PC of molecular variability. E.g., in some embodiments, fractions of the lipids PE and PtdCho have the largest loadings, and the ratio between the identified fractions as it better characterizes individual brain regions.

In some embodiments, the method is used to identify two other features with large loadings, e.g., the fraction of the lipid Spg and the phospholipids/proteins ratio and predict these 3 human brain molecular features using the MTV derivatives to account for most of the MDM variability. E.g., two measurements with the largest loadings on the first PC of MDM are dR1/dMTV and dMTsat/dMTV. Therefore, in the case of the human brain, F is a 3×7 matrix of molecular composition estimated from the literature. The columns of F are seven different brain areas. The rows of F are the three molecular features with largest loadings. Mmix is a 2×7 matrix of the MDM measurements of seven different brain regions. In this example, the data was calculated from the MRI measurements averaged over the young subjects. The rows of Mmix are two MDM measurements with large loadings on the first PC of MDM variability.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

Methods and computer program products are disclosed herein that may automatically construct (i.e., without human intervention) a list of relevant claims and supportive evidence given a topic under consideration (TUC). Thus, for example, one may extract persuasive claims supporting his or her point of view as well as be prepared for counter claims which the other side may raise while discussing the TUC.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method for quantification of a molecular composition of a scanned tissue based on an MM signal comprising:
receiving qMRI (quantitative MM) data from a tissue scan, the qMRI data comprising at least a first parameter and a second parameter, wherein said first parameter is a non-water fraction of the scanned tissue;
calculating a dependency of said second parameter on said first parameter; and
quantifying a molecular composition of said scanned tissue based on said calculation.

2. The method according to claim 1, wherein said tissue is brain tissue.

3. The method according to claim 2, wherein associating molecular composition of said scanned tissue with at least one of a specific region of the brain, a specific pathology of said brain tissue and an age of said scanned tissue.

4. The method according to claim 1, wherein eliminating the confounding effect of water on said qMRI parameters by said evaluating a dependency of said qMRI parameters on a non-water fraction of said scanned tissue.

5. The method according to claim 1, wherein said obtained qMRI parameters comprise at least one of T1, T2, R1, R2, MT, MTV, susceptibility and CEST.

6. The method according to claim 1, wherein said non-water fraction of said scanned tissue is at least Proton Density (PD).

7. The method according to claim 1, wherein quantifying the MTV to overcome the confounding effect of water content on qMRI parameters and deriving therefrom tissue-specific properties.

8. The method according to claim 7, wherein generating for at least one brain area of different brain areas a linear fit the slope of which represents MTV derivatives of one of R1 and MTsat.

9. (canceled)

10. The method according to claim 7, wherein associating changes in said non-water fraction with changes in a molecular composition of a tissue being scanned.

11. The method according to claim 7, wherein the tissue is brain tissue and said method comprises associating changes in said non-water fraction with specific regions of the brain being scanned.

12. The method according to claim 7, wherein associating changes in said non-water fraction with change in age of the tissue being scanned.

13. The method according to claim 1, wherein said tissue is brain tissue and said method comprises generating a plurality of a unique tissue signatures for corresponding brain regions over a period of time and measuring for each signature the dependency of qMRI parameters on MTV.

14. The method according to claim 1, wherein said tissue is brain tissue and said method comprises combining local dependencies of said different qMRI parameters on MTV and generating a unique signature for at least one corresponding brain region from said different brain regions.

15. The method according to claim 14, wherein identifying and classifying aberrant scan signatures of specific brain regions as qMRI scan signatures corresponding to specific pathologies in said specific brain regions.

16. (canceled)

17. A method for predicting the molecular composition of the human brain, said method comprising:
estimating a matrix Mpure for a selected region of brain tissue;
measuring MDM measurement of lipid mixture in said region and deriving Mmix where Mmix is a vector of said MDM measurements, such that:
[Mₘᵢₓ]=[Mₚᵤᵣₑ]*[F]
wherein F is a vector of the lipid fractions of the mixture, and Mpure is a matrix of the MDM measurements of the pure lipids; and
estimating the lipid composition of the brain tissue in said brain segment by calculating
[F]=[Mpure]−1*[Mmix].

18. The method according to claim 17, wherein the calculation includes at least one of human brain molecular features: % PE, % PS, % PtdCho % PI, % Spg, phospholipids/proteins and phospholipids/cholesterol.

19. The method according to claim 17, wherein the calculation includes at least one of MDM measurements: dR1/dMTV, dMTsat/dMTV, dR2dMTV, dMD/dMTV.

20. The method according to claim 17, wherein the method comprises identifying a plurality of molecular features with the largest loadings on the first PC of molecular variability.

21. A computer program product comprising a non-transitory computer-readable storage medium having program code embodied therewith, the program code executable by at least one hardware processor to:
receive qMRI (quantitative MRI) data from a tissue scan, the qMRI data comprising at least a first and a second parameter, wherein said first parameter is a non-water fraction of the scanned tissue;
calculate a dependency of said second qMRI parameter on a non-water fraction of said scanned tissue; and
quantify a molecular composition of said scanned tissue based on said calculation.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
